(19) United States
(12) Patent Application Publication
(10) Pub. No.: US 2023/0028683 A1
(43) Pub. Date: Jan. 26, 2023

(54) BIODEGRADABLE MICROCAPSULE SYSTEMS

(71) Applicant: KOEHLER PAPER SE, Oberkirch (DE)

(72) Inventors: Christian KIND, Baden-Baden (DE); Jeanette HILDEBRAND, Bühl (DE); Klaus LAST, Braunschweig (DE); Claudia MEIER, Lichtenau (DE)

(73) Assignee: KOEHLER PAPER SE, Oberkirch (DE)

(21) Appl. No.: 17/783,541

(22) PCT Filed: Dec. 11, 2020
(86) PCT No.: PCT/EP2020/085804

§ 371 Date: Jun. 8, 2022

(30) Foreign Application Priority Data
Dec. 12, 2019 (EP) PCT/EP2019/084894

(51) Int. Cl. B01J 13/14; C11D 3/00; B01J 13/22; C11D 3/50

(52) U.S. Cl. CPC B01J 13/14; C11D 3/001; B01J 13/22; C11D 3/505

(57) ABSTRACT

In accordance with a first aspect, the invention relates to microcapsules for use in a high demand area selected from detergents and cleaners, cosmetic products, adhesive systems, paints and dispersions, coating materials comprising a core material and a shell, wherein the shell consists of at least a first and a second layer whose chemical compositions differ, and wherein the shell has a biodegradability measured according to OECD 301 F of at least 40%. The invention further relates to a product comprising microcapsules, wherein the product is selected from the group consisting of an adhesive system; a cosmetic product; a pharmaceutical product; a coating material, in particular a coated paper; a heat storage coating, a self-healing coating or a corrosion coating; and a coating of functional packaging materials.

### FIELD OF THE INVENTION

The invention relates to stable microcapsules with environmentally acceptable wall materials for use in application fields with high requirements for the impermeability and stability of the microcapsules.

### BACKGROUND OF THE INVENTION

Microencapsulation is a versatile technology. It offers solutions for numerous innovations—from the paper industry to household products, microencapsulation increases the functionality of a wide range of active substances. Encapsulated active substances can be used more economically and improve the sustainability and environmental compatibility of many products

However, the polymeric wall materials of the microcapsules themselves are environmentally compatible to very different degrees. Microcapsule walls based on the natural product gelatin and thus completely biodegradable have long been used in carbonless paper. A gelatin encapsulation process developed as early as the 1950s is disclosed in U.S. Pat. No. 2,800,457. Since then, a variety of variations in terms of materials and process steps have been described. In addition, biodegradable or enzymatically degradable microcapsule walls are used to employ enzymatic degradation as a method for releasing the core material. Such microcapsules are described, for example, in WO 2009/126742 A1 or WO 2015/014628 A1.

However, such microcapsules are not suitable for many industrial applications and household products. This is because natural-substance-based microcapsules do not meet the diffusion impermeability, chemical resistance and temperature resistance required for detergents and cleaning agents, adhesive systems, coatings and dispersions, for example, nor the required loading with core material.

In these so-called high-demand areas, classically organic polymers such as melamine-formaldehyde polymers (see e.g., EP 2 689 835 A1, WO 2018/114056 A1, WO 2014/016395 A1, WO 2011/075425 A1 or WO 2011/120772 A1); Polyacrylates (see e.g., WO 2014/032920 A1, WO 2010/79466 A2); Polyamides; Polyurethane or polyureas (see e.g., WO 2014/036082 A2 or WO 2017/143174 A1).

The capsules constructed of such organic polymers have the required diffusion impermeability, stability and chemical resistance. However, these organic polymers are only enzymatically or biodegradable to a very limited extent.

Various approaches are described in the prior art, in which biopolymers are combined as an additional component with the organic polymers of the microcapsule shell for application in high demand areas, but not with the objective to produce biodegradable microcapsules, but priority the releasing, stability, or surface properties to change the microcapsules. For example, in WO 2014/044840 A1, a method for producing two-layered microcapsules described with an inner polyurea layer and an outer gelatin-containing layer. The polyurea layer is generated by polyaddition on the inside of the gelatin layer obtaining by coacervation. The capsules thus obtained have the necessary stability and impermeability for use in detergents and cleaning agents due to the polyurea layer and additionally by the gelatin to attach them to surfaces. Concrete stabilities and resistance are not mentioned. A disadvantage of poly urea capsules, however, is the inevitable side reaction of the core materials having the diisocyanates used to produce the urea, which must be added to the oil-based core.

On the other hand, biopolymer-based microcapsules are also described in the prior art, which achieve improved impermeability or stability against environmental influences or a targeted setting of a delayed release behavior by adding a protective layer. For example, WO 2010/003762 A1 describes particles with a core-shell-shell structure. Inside each particle there is a poorly water-soluble or water-insoluble organic active ingredient as a core. The shell directly surrounding the core contains a biodegradable polymer and the outer shell contains at least one metal or semimetal oxide. With this structure, a biodegradable shell is indeed obtained. Nevertheless, the microcapsules are used in food, cosmetics or pharmaceuticals according to WO 2010/003762 A1, but are not usable for the high demand areas according to the invention due to lack of impermeability.

### SUMMARY OF THE INVENTION

The present invention is based, inter alia, on the discovery that by means of a multilayer shell structure, microcapsules can be produced that are substantially biodegradable and yet have sufficient stability and impermeability to be used in high-demand applications such as detergents and cleaning agents. This is achieved by having a first stability- and structure-imparting layer make up the bulk of the capsule shell, consisting of naturally occurring and readily biodegradable materials, such as gelatin or alginate, or materials ubiquitously found in nature. This first layer is combined with a second density-imparting layer, which may consist of known materials used for microencapsulation, such as melamine-formaldehyde or meth(acrylate). The second layer may be located on the outside of the first layer as well as on the inside of the first layer. Preferably, the second layer is arranged on the inner side of the first layer. The inventors have succeeded in designing the impermeability-imparting second layer with a previously unimaginable low wall thickness and yet still ensuring sufficient impermeability, as shown in Example 5. Thus, the proportion of the total wall is kept very low, so that the microcapsule wall has a biodegradability measured according to OECD 301 F of at least 40% as shown in examples 6 and 7.

Thus, according to a first aspect, the invention relates to microcapsules for use in a high demand area selected from detergents and cleaners, cosmetic products, adhesive systems, paints and dispersions, coating materials comprising a core material and a shell, wherein the shell consists of at least a first and a second layer whose chemical compositions differ, and wherein the shell has a biodegradability measured according to OECD 301 F of at least 40%.

Due to the robustness or impermeability of this biodegradable capsule, it can be used in a variety of products. Consequently, according to a second aspect, the invention relates to a product comprising microcapsules according to the first aspect wherein the product is selected from the group consisting of an adhesive system; a cosmetic product; a pharmaceutical product; a coating material, in particular a coated paper; a heat storage coating, a self-healing coating or a corrosion coating; and such microcapsule-containing coatings for functional packaging materials.

Further, in a third aspect, the invention relates to the use of microcapsules according to the first aspect for producing a product according to the second aspect.
Finally, in a fourth aspect, the invention relates to a method for producing microcapsules according to the first aspect 1, characterized by the following steps:
a) preparing an oil-in-water emulsion by emulsifying a core material in an aqueous phase in the presence of the wall-forming component(s) of the inner, second shell layer, optionally with the addition of protective colloids;
b) depositing and curing the wall-forming component(s) of the inner, second shell layer, wherein the wall-forming component(s) of the inner, second shell layer are in particular an aldehydic component, an amine component and an aromatic alcohol;
c) addition of the wall-forming component(s) of the middle, first shell layer, followed by deposition and curing, the wall-forming component(s) of the middle, first shell layer being in particular proteins and/or polysaccharides; and
d) optionally addition of the wall-forming component(s) of the outer, third shell layer, followed by deposition and curing, the wall-forming component(s) of the outer, third shell layer being in particular an amine component; and the microcapsules obtained thereby.

### FIGURES

FIG. 1 shows a light microscopic image of the capsules MK 1 according to the invention at a magnification of 50× and a magnification of 500× taken with an Olympus BX 50 microscope.

FIG. 2 shows a light microscopic image of the reference microcapsule MK 2 (melamine-formaldehyde) at a magnification of 50× and 500× taken with an Olympus BX 50 microscope.

FIG. 3 shows a light microscopy image of the reference microcapsule MK 3 (gelatin alginate) at a magnification of 50× and 500× taken with an Olympus BX 50 microscope.

FIG. 4 shows a diagram of the course of biodegradation of the microcapsule MK 1 according to the invention over 28 days (shown as a solid line) (a) shows the result according to OECD301F. As a positive control, the degradation of ethylene glycol is shown as a dashed line (b) shows the result according to OECD302C. As a positive control, the degradation of aniline is shown in the form of a dashed line.

FIG. 5 shows a comparison of the course of biodegradation over 28 days of the microcapsule MK 1 according to the invention, the MF reference microcapsule MK 2 and the gelatin/alginate reference microcapsule MK 3. Shown is a measurement according to OECD301F for the first 10 days of biodegradation. Furthermore, the time window in which the microcapsule MK 1 according to the invention reaches a degradation level of 60% is shown.

FIG. 6 shows a light microscopic image of the capsules MK 4 according to the invention at a 50× and a 500× magnification taken with an Olympus BX 50 microscope.

FIG. 7 shows a diagram of the course of biodegradation according to OECD 301 F over 60 days after washing the microcapsule MK 1 according to the invention over time as well as the MF reference microcapsule MK 2 and the gelatin/alginate reference microcapsule MK 3. As a positive control, both the degradation of ethylene glycol is shown in the form of a dashed line and the degradation of walnut shell flour is shown in the form of a dotted line.

### DETAILED DESCRIPTION OF THE INVENTION

### Definitions

“Biodegradability” refers to the ability of organic chemicals to be biologically decomposed, that is, by living organisms or their enzymes. Ideally, this chemical metabolism proceeds completely to mineralization, but may stop at degradation-stable transformation products. Generally accepted are the guidelines for testing chemicals of the OECD, which are also used in the context of chemical approval. The tests of the OECD Test Series 301 (A-F) demonstrate rapid and complete biodegradation (ready biodegradability) under aerobic conditions. Different test methods are available for well or poorly soluble as well as for volatile substances. In particular, the manometric respiration test (OECD 301 F) is used within the scope of the application. The basic biodegradability (inherent biodegradability) can be determined via the measurement standard OECD 302, for example the MITI I I test (OECD 302 C).

“Biodegradable” or “biodegradable” in the sense of the present invention refers to microcapsule walls that have a biodegradability measured according to OECD 301 F of at least 40% or measured according to OECD 302 C (MITI-II test) of at least 20% and thus exhibit inherent or fundamental degradability. This corresponds to the limit value for OECD 302 C according to “Revised Introduction to the OECD Guidlines for testing of Chemicals, section 3, Part 1, dated 23 Mar. 2006”. From a limit value of at least 60% measured according to OECD 301 F, microcapsule walls are also referred to here as rapidly biodegradable.

“Impermeability” to a substance, gas, liquid, radiation or the like, is a property of material structures. According to the invention, the terms “impermeability” and “tightness” are used synonymously. Impermeability is a relative term and always refers to given general conditions.

“High demand areas” in the sense of the invention are application areas with high demands on the impermeability and stability of the microcapsules.

The term “(meth)acrylate” in the present invention refers to both methacrylates and acrylates.

According to the invention, the term “microcapsules” refers to particles containing an inner space or core filled with a solid, gelled, liquid or gaseous medium and enclosed (encapsulated) by a continuous coating (shell) of film-forming polymers. These particles preferably have small dimensions. The terms “microcapsules,” “core-shell capsules,” or simply “capsules” are used interchangeably.

“Microencapsulation” refers to a manufacturing process in which small and miniscule portions of solid, liquid or gaseous substances are surrounded by a shell of polymeric or inorganic wall materials. The microcapsules thus obtained may have a diameter of a few millimeters to less than 1 μm.

The microcapsule according to the invention thus has a multilayer shell. The shell encapsulating the core material of the microcapsule is also regularly referred to as the “wall” or “sheath.”

The microcapsules according to the invention with a multilayer shell can also be referred to as multilayer microcapsules or multilayer microcapsule system, since the individual layers can also be regarded as individual shells. “Multilayer” and “multishell” are thus used interchangeably.

Wall formers” are the components that build up the microcapsule wall. Microcapsules

According to a first aspect, the invention relates to microcapsules comprising a core material and a shell, wherein the shell comprises at least a first and a second layer whose chemical compositions differ, and wherein the shell has a biodegradability measured according to OECD 301 F of at least 40%. As measured by OECD 302 C, the microcapsules of the invention have a biodegradability of at least 20%.

As shown in Examples 6 and 7, the microcapsule shells according to the invention are biodegradable according to OECD due to the high content of natural components.

According to one embodiment, the first layer of the microcapsules contains one or more biodegradable components as wall formers. This first layer forms the main stability-imparting component of the microcapsule shell and thus ensures the high biodegradability according to OECD 301 F of at least 40%. Biodegradable components suitable as wall formers for the first layer are proteins such as gelatin; polysaccharides such as alginate, gum arabic, chitin, or starch; phenolic macromolecules such as lignin; polyglucosamines such as chitosan, polyvinyl esters such as polyvinyl acetate and polyvinyl alcohols, in particular highly saponified and fully saponified polyvinyl alcohols; phosphazenes and polyesters such as polylactide or polyhydroxyalkanoate. This list of specific components in the individual substance classes is only exemplary and should not be understood as limiting. Suitable natural wall formers are known to the skilled person. Furthermore, the skilled person is familiar with the various processes for wall formation, for example coacervation or interfacial polymerization.

These biodegradable components can be appropriately selected for the particular application to form a stable multilayer shell with the material of the second layer. The second layer may be located on the outside of the first layer as well as on the inside of the first layer. Preferably, the second layer is disposed on the inner side of the first layer. In addition, the biodegradable components can be selected to ensure, for example, if arranged on the inner side, compatibility with the core material or—if arranged on the outer side—to achieve compatibility with the chemical conditions of the application area. The biodegradable components can be combined in any way to affect the biodegradability or also, for example, stability and chemical resistance of the microcapsule.

In one embodiment of the first aspect, the shell of the microcapsules has a biodegradability of 50% according to OECD 301 F. In another embodiment, the shell of the microcapsule has a biodegradability of at least 60% (OECD 301 F). In a further embodiment, the biodegradability is at least 70% (OECD 301 F). According to OECD 302 C, the microcapsule according to the invention may have a biodegradability of at least 25%. According to one embodiment, the biodegradability is at least 30% (OECD 302 C). According to a further embodiment, the biodegradability is at least 40% (OECD 302 C). In each case, the biodegradability is measured over a period of 28 days. In the extended degradation method (“enhanced ready biodegredation”), biodegradability is measured over a period of 60 days (see Opinion on an Annex XV dossier proposing restrictions on intentionally added microplastics of Jun. 11, 2020 ECHA/RAC/RES-0-00006790-71-01/F). Preferably, the microcapsules are washed to remove dissolved residues prior to the determination of biodegradability. In one embodiment, the capsule dispersion is washed after preparation by centrifuging and redispersing three times in water. For this purpose, the sample is centrifuged. After aspiration of the clear supernatant, water is added, and the sediment is redispersed by shaking. When measuring the biodegradability, various reference samples can be used, such as the rapidly degradable ethylene glycol or natural-based walnut shell flour with the typical step-like degradation of a complex mixture of substances. The microcapsule according to the invention shows a similar, preferably better biodegradability over a period of 28 or 60 days than the walnut shell flour.

A high value of biodegradability according to the invention is achieved on the one hand by the wall formers used and on the other hand by the structure of the shell according to the invention. This is because the use of a certain percentage of natural potentially biodegradable components does not automatically lead to a corresponding value of biodegradability. This is dependent on how the potentially biodegradable components are present in the shell.

According to a preferred embodiment, the first layer comprises gelatin. According to another preferred embodiment, the first layer comprises alginate. According to a further preferred embodiment, the first layer comprises gelatin and alginate. As shown in the embodiment, both gelatin and alginate are suitable for the production of microcapsules according to the invention with high biodegradability and high stability. Other suitable combinations of natural components in the first layer include gelatin and gum arabic.

A high value of biodegradability according to the invention is achieved on the one hand by the wall formers used and on the other hand by the structure of the shell according to the invention. This is because the use of a certain percentage of natural potentially biodegradable components does not automatically lead to a corresponding value of biodegradability. This is dependent on how the potentially biodegradable components are present in the shell.

According to a preferred embodiment, the first layer comprises gelatin. According to another preferred embodiment, the first layer comprises alginate. According to a further preferred embodiment, the first layer comprises gelatin and alginate. As shown in the embodiment, both gelatin and alginate are suitable for the production of microcapsules according to the invention with high biodegradability and high stability. Other suitable combinations of natural components in the first layer include gelatin and gum arabic.

According to one embodiment, the first layer comprises one or more curing agents. Curing agents according to the invention include aldehydes such as, for example, glutaraldehyde, formaldehyde, and glyoxal, as well as tannins, enzymes such as transglutaminase, and organic anhydrides such as maleic anhydride. Preferably, the curing agent is glutaraldehyde due to its very good crosslinking property. Further preferred is the curing agent glyoxal due to its good crosslinking properties and, compared to glutaraldehyde, lower toxicological classification. By using curing agents, a higher impermeability of the first layer consisting of natural murals is achieved. In addition, curing agents reduce the tackiness of the layer and thus the tendency to agglomeration. However, curing agents lead to reduced biodegradability of the natural polymers. Due to the combination of the first layer with the second layer as a diffusion barrier, the amount of curing agent in the first layer can be kept low, which in turn contributes to the easy biodegradability of the layer. According to one embodiment, the amount of curing agent in the first layer is below 25% by weight. Unless explicitly defined otherwise, the proportions of the components of the layers refer to the total weight of the layer, i.e., the total dry weight of the components used in the production, excluding the components used in the production that are not or only slightly incorporated into the layer, such as surfactants and protective colloids. Above this value, the biodegradability according to OECD 301 F of the invention cannot be guaranteed. Preferably, the proportion of curing agent of the first layer is in the range of 5-15% by weight. This proportion leads to effective crosslinking of the gelatin and, in a quantitative reaction, results in as little residual monomer as possible. The range 9 to 12 wt % is particularly preferred, it provides the required degree of crosslinking and stable coating of the second shell to buffer the otherwise sensitive diffusion barrier and provide it with further barrier properties and has little residual aldehyde, which is degraded via an aldol reaction in a downstream alkaline adjustment of the slurry.

In one embodiment, the first layer contains gelatin and glutaraldehyde. According to another embodiment, the first layer comprises gelatin, alginate, and glutaraldehyde. According to an additional embodiment, the first layer comprises gelatin and glyoxal. According to a further embodiment, the first layer comprises gelatin, alginate and glyoxal. The exact chemical composition of the first layer is not critical. It only has to ensure sufficient stability of the microcapsule wall and the release behavior required for the particular application. What is essential is that it contains only small amounts or preferably no unnatural persistent components. Consequently, the first layer may also contain one or more inorganic components as wall formers as an alternative or in addition to the biodegradable components. Inorganic components as wall formers can be, in particular, calcium carbonates or polysilicates. These are particularly suitable because, as ubiquitous components, they are environmentally friendly. Since there is therefore no need to degrade these inorganic components, they are regarded as completely biodegradable according to the invention, even if the criteria according to OECD 301 or OECD 302 are not applicable to these components.

According to the invention, the second layer is also referred to as a density-imparting layer or diffusion barrier. This is because, despite the thin wall thickness of the second layer, the microcapsules exhibit high impermeability. As shown in Example 5, the impermeability is sufficient for use in a flooring application. According to one embodiment, the second layer has an average thickness in the range of 0.01 μm to 1 μm. A higher layer thickness than 1 μm would increase the proportion of the components of the second layer in the total capsule wall too much and thus no longer ensure sufficient biodegradability. With a layer thickness of less than 0.01 μm, the second layer would no longer be a sufficient diffusion barrier.

Thus, the microcapsules would be unsuitable for high-demand areas. With a layer thickness of 0.02 μm or more, the second layer has sufficient impermeability for most applications. For easy biodegradability of the microcapsule, the wall thickness of the second layer should be 0.5 μm or less. Particularly preferably, the wall thickness of the second layer is in the range of 0.05 μm to 0.30 μm. In this range, an optimum density is achieved with easy biodegradability.

The second layer preferably contains as wall former one or more components selected from the group consisting of an aldehydic component, an aromatic alcohol, an amine component, an acrylate component. Manufacturing processes for producing microcapsules with these wall materials are known to the skilled person. A polymer selected from a polycondensation product of an aldehydic component with one or more aromatic alcohols, and/or amine components may be used to prepare the second layer.

As shown in embodiments 1 and 4, the low wall thickness of the second layer according to the invention can be achieved in particular with a melamine-formaldehyde layer comprising aromatic alcohols or m-aminophenol. Consequently, the second layer preferably comprises an aldehydic component, an amine component, and an aromatic alcohol.

The use of amine-aldehyde compounds in the second layer, in particular melamine-formaldehyde, has the advantage that these compounds form a hydrophilic surface with a high proportion of hydroxy functionality, which thus exhibit excellent compatibility with hydrogen bond oriented components of the first layer, such as biodegradable proteins, polysaccharides, chitosan, lignins and phosphazenes but also inorganic wall materials such as CaCO3 and polysiloxanes. In the same way, polyacrylates, in particular from the components styrene, vinyl compounds, methyl methacrylate, and 1,4-butanediol acrylate, methacrylic acid, can be generated by initiation, for example with t-butyl hydroperoxide in a free radical induced polymerization (polyacrylates) as microcapsule wall, which form a hydrophilic surface with a high proportion of hydroxy functionality, which are therefore just as compatible with the components of the first layer according to the invention.

Thus, in a preferred embodiment, a wall former of the second layer is an aldehydic component. According to one embodiment, the aldehydic component of the second layer is selected from the group consisting of formaldehyde, glutaraldehyde, succinaldehyde, furfural and glyoxal. Microcapsules have already been successfully produced with all these aldehydes (see WO 2013 037 575 A1), so it can be assumed that similar dense capsules can be obtained with them as with formaldehyde.

Based on the studies of the present invention, the proportion of the aldehydic component for wall formation relative to the total weight of the second shell should be in the range of 5% w/w to 50% w/w. It is expected that outside these limits, it is not possible to obtain a sufficiently stable and dense thin layer. Preferably, the concentration of the aldehydic component in the second layer is in the range of 10% w/w to 30% w/w. Particularly preferably, the concentration of the aldehydic component in the second layer is in the range of 15% w/w to 20% w/w.

Suitable amine components in the second layer include, in particular, melamine, melamine derivatives and urea or combinations thereof. Suitable melamine derivatives include etherified melamine derivatives and methylolated melamine derivatives. Melamine in the methylolated form is preferred. The amine components can be used, for example, in the form of alkylated mono- and polymethylol-flurea precondensation products or partially methylolated mono- and polymethylol-1,3,5 triamono-2,4,6 triazine precondensation products such as Luracoll SD® (from BASF). According to one embodiment, the amine component is melamine. According to an alternative embodiment, the amine component is a combination of melamine and urea.

The aldehydic component and the amine component may be present in a molar ratio ranging from 1:5 to 3:1. For example, the molar ratio may be 1:5, 1:4.5, 1:4, 1:3.5, 1:3, 1:2.5, 1:2, 1:1.8, 1:1.6, 1:1.4, 1:1.3, 1:1.2, 1:1, 1.5:1, 2:1, 2.5:1, or 3:1. Preferably, the molar ratio is in the range of 1:3 to 2:1. Particularly preferably, the molar ratio of the aldehydic component and the amine component can be in the range of 1:2 to 1:1. The aldehydic component and the amine component are generally used in a ratio of about 1:1.3. This molar ratio allows a complete reaction of the two reactants and leads to a high impermeability of the capsules. There are also known, for example, aldehyde-amine capsule walls with a molar ratio of 1:2. These capsules have the advantage that the proportion of the highly crosslinking aldehyde, in particular formaldehyde, is very low. However, these capsules have a lower impermeability than the capsules with a ratio of 1:1.3. Capsules with a ratio of 2:1 have an increased impermeability, but have the disadvantage that the aldehyde component is partially present unreacted in the capsule wall and the slurry.

In one embodiment, the proportion of amine components (e.g., melamine and/or urea) in the second layer relative to the total weight of the second layer is in the range of 20% w/w to 85% w/w. For example, the proportion of the amine component may be 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, or 85% w/w. In a preferred embodiment, the proportion of the amine component in the second layer relative to the total weight of the second layer is in the range from 40 wt. % to 80 wt. %. Particularly preferably, the proportion of the amine component is in the range of 55% w/w to 70% w/w.

With the aromatic alcohol, it is possible to greatly reduce the wall thickness of the second layer built up from the amine component and the aldehyde component in order to nevertheless obtain a layer that has the necessary impermeability and is stable enough at least in combination with the first layer. The aromatic alcohols impart increased impermeability to the wall, since their highly hydrophobic aromatic structure makes it difficult for low molecular weight substances to diffuse through. As shown in the examples, phloroglucin, resorcinol or m-aminophenol are particularly suitable aromatic alcohols. Consequently, in one embodiment, the aromatic alcohol is selected from the group consisting of phloroglucin, resorcinol and aminophenol. In combination with the amine component and the aldehyde component, the aromatic alcohol is formed in a molar ratio to the aldehyde component in the range of (alcohol:aldehyde) 1:1 to 1:20, preferably in the range of 1:2 to 1:10.

In one embodiment, the proportion of the aromatic alcohol in the second layer based on the total weight of the second layer is in the range of 1.0% w/w to 20% w/w. For example, the proportion of aromatic alcohol may be 1.5% w/w, 2.0% w/w, 2.5% w/w, 3.0% w/w, 4.0% w/w, 5.0% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, or 20% w/w. Due to their aromatic structure, the aromatic alcohols impart a coloration to the capsule wall that increases with the proportion of aromatic alcohol. Such coloration is undesirable in a variety of applications. In addition, the aromatic alcohols are susceptible to oxidation, which leads to a change in coloration over time. As a result, the undesirable coloration of the microcapsules can be poorly compensated with a dye. Therefore, the aromatic alcohols should not be used above 20.0 w/w. Below 1.0% w/w by weight, no effect is detectable with respect to impermeability. In a preferred embodiment, the percentage of the aromatic alcohol in the second layer, based on the total weight of the second layer, is in the range of 5.0% w/w to 15.0% w/w. Up to a percentage of 15.0 wt %, the coloration is tolerable in most applications. In a particularly preferred embodiment, the percentage of aromatic alcohol in the second layer based on the total weight of the second layer is in the range of 7.0% w/w to 13.0% w/w. In particular, the proportion of the aromatic alcohol in the second layer is in the range of 9.0% w/w to 13.0% w/w.

In a further embodiment, the aldehyde component of the second layer may be used together with an aromatic alcohol such as resorcinol, phloroglucin, or m-aminophenol as the wall-forming component(s), i.e., omitting the amine component(s).

In one embodiment, the second layer of the microcapsules contains melamine, formaldehyde, and resorcinol. In one embodiment, the second layer of the microcapsules comprises melamine, urea, formaldehyde, and resorcinol. In a preferred embodiment, the second layer of the microcapsules contains melamine in the range of 25 to 40% w/w, formaldehyde in the range of 15 to 20% w/w, and resorcinol in the range of 0.1 to 12% w/w, and optionally urea in the range of 15 to 20% w/w. The proportions refer to the amounts used for the wall formation of the layer and are based on the total weight of the second layer without protective colloid.

A protective colloid may further be used to prepare the second layer from an aldehydic component, an amine component and an aromatic alcohol. A suitable protective colloid is 2-acrylamido-2-methyl-propanesulfonic acid (AMPS, commercially available as Lupasol® PA 140, BASF) or its salts. The proportion of the protective colloid in the components used to produce the second layer can be in the range of 10 to 30% w/w based on the total dry weight of the components used. According to one embodiment, the proportion of the protective colloid in the components used to produce the second layer is in the range of 15 to 25% w/w. The protective colloid may also be present at a certain low percentage in the finished microcapsule shell. Determining the percentage of the protective colloid in the second layer is technically difficult. In addition, the percentage is only small.

Consequently, the other proportions of the other components are presented as if the protective colloid were not included.

The (meth)acrylate polymers optionally used for forming the thin second layer (diffusion barrier) may be homo- or copolymers of methacrylate monomers and/or acrylate monomers. The (meth)acrylate polymers are, for example, homo- or copolymers, preferably copolymers, of one or more polar functionalized (meth)acrylate monomers, such as sulfonic acid group-containing, carboxylic acid group-containing, phosphoric acid group-containing, nitrile group-containing, phosphonic acid-containing, ammonium group-containing, amine group-containing or nitrate group-containing (meth)acrylate monomers. The polar groups can also be present in salt form.

(Meth)acrylate copolymers may, for example, consist of two or more (meth)acrylate monomers (e.g., acrylate+2-acrylamido-2-methyl-propanesulfonic acid) or of one or more (meth)acrylate monomers and one or more monomers other than (meth)acrylate monomers (e.g., methacrylate+styrene).

Examples of (meth)acrylate polymers include homopolymers of sulfonic acid group-containing (meth)acrylates (e.g. 2-acrylamido-2-methyl-propanesulfonic acid or its salts (AMPS), or their copolymers, copolymers of acrylamide and (meth)acrylic acid, copolymers of alkyl (meth)acrylates and N-vinylpyrrolidone (commercially available as Luviskol® K15, K30 or K90, BASF), copolymers of (meth)acrylates with polycarboxylates or polystyrenesulfonates, copolymers of (meth)acrylates with vinyl ethers and/or maleic anhydride, copolymers of (meth)acrylates with ethylene and/or maleic anhydride, copolymers of (meth)acrylates with isobutylene and/or maleic anhydride, or copolymers of (meth)acrylates with styrene-maleic anhydride.

Preferred (meth)acrylate polymers are homopolymers or copolymers, preferably copolymers, of 2-acrylamido-2-methyl-propanesulfonic acid or salts thereof (AMPS). Preferred are copolymers of 2-acrylamido-2-methyl-propanesulfonic acid or its salts, e.g. copolymers with one or more comonomers from the group of (meth)acrylates, vinyl compounds such as vinyl esters or styrenes, unsaturated di- or polycarboxylic acids such as maleic esters, or salts of amyl compounds or allyl compounds.

In contrast to known biodegradable microcapsules, the microcapsules according to the invention exhibit a high degree of impermeability. According to one embodiment, the microcapsules have an impermeability that ensures a leakage of at most 80% by weight of the core material used after storage for a period of 12 weeks at a temperature of 0 to 40° C.

In addition to the shell material, the impermeability also depends on the type of core material. The impermeability of the microcapsules according to the invention was determined in accordance with the invention for the fragrance oil Weiroclean from Kitzing, since this fragrance oil is representative in its chemical properties for microencapsulated fragrance oils. Weiroclean has the following components (with proportion based on total weight):

| | |
|---|---|
| 1-(1,2,3,4,5,6,7,8-Octahydro-2,3,8,8-tetramethyl- | 25-50% |
| 2-naphthalenyl)ethanone | |
| Benzoic acid, 2-hydroxy, 2-hexyl ester | 10-25% |
| Phenyl methyl benzoate | 5-10% |
| 3-Methyl-4-(2,6,6-trimethyl-2-cyclohexenyl)-3-buten-2-on | 1-5% |
| 3,7-Dimethyl-6-octen-1-ol | 1-5% |
| 3-Methyl-5-phenylpentanol | 1-5% |
| 2,6-Dimethyloct-7-en-2-ol | 1-5% |
| 4-(2,6,6-Tri methylcyclohex-1-eneyl)-but-3-ene-2-one | 1-5% |
| 3a,4,5,6,7,7a-Hexahydro-4,7-methano-1H-inden-6-yl | 1-5% |
| propanoate | |
| 2-tert-Butylcyclohexyl acetate | 1-5% |
| 2-Heptylcyclopentanone | 1-5% |
| Pentadecane-15-olide | 1-5% |
| 2H-1-Benzopyran-2-one | 0.1-1% |
| 2,6-Di-tert-butyl-p-cresol | 0.1-1% |
| 4-Methyl-3-decen-5-ol | 0.1-1% |
| 2,4-Dimethyl-3-cyclohexene-1-carboxaldehyde | 0.1-1% |
| [(2E)-3,7-Dimethylocta-2,6-dienyl]acetate | 0.1-1% |
| Allyl hexanoate | 0.1-1% |
| 2-Methylundecanal | 0.1-1% |
| 10-Undecenal 0.1-1% cis-3,7-dimethyl-2,6-octadienyl | 0.1-1% |
| ethanoate | |
| 3,7,11-trimethyldodeca-1,6,10-trien-3-ol | 0.1-1% |
| Undecane-2-one | 0.1-1% |

A variety of different materials can be used as core materials, including fragrances, flavors, phase change materials, cosmetic active ingredients, pharmaceutical active ingredients, catalysts, initiator systems, adhesive components and hydrophobic reactive components. According to a preferred embodiment of the microcapsules of the invention, the core material is hydrophobic. The core material may be solid or liquid. In particular, it is liquid. Preferably, it is a liquid hydrophobic core material. In a preferred embodiment, the core material is a fragrance. Particularly preferred are fragrance oils optimized for microencapsulation for the detergent and cleaning agent sector, such as the fragrance formulation Weiroclean (Kurt Kitzing GmbH).

The impermeability of the capsule wall can be influenced by the choice of shell components. According to one embodiment, the microcapsules have an impermeability that ensures a leakage of at most 75% by weight, at most 70% by weight, at most 65% by weight, at most 60% by weight, at most 55% by weight, at most 50% by weight, at most 45% by weight, at most 40% by weight of the core material used when stored for a period of 12 weeks at a temperature of 0 to 40° C. In this process, the microcapsules are stored in a model formulation corresponding to the target application. Furthermore, the microcapsules are also storage stable in the product in which they are used. For example, in detergents, fabric softeners, cosmetic products, adhesive systems, coatings and dispersions, or in layered materials, such as coated papers. The guide formulations of these products are known to the skilled person. Typically, the pH value in the environment of the microcapsules during storage is in the range of 2 to 10.

The second layer may be disposed on the inner or outer surface of the first layer. According to one embodiment, the second layer is arranged on the inner side of the first layer. Such an arrangement has the advantage that the density-imparting layer can additionally serve as a chemical protective layer between the biodegradable first layer and the core material. This is especially important in cases where the core material may chemically attack the biodegradable material of the first layer. The problem with this structure is that the very thin second layer must first be formed as a template during encapsulation. This was solved in the present case by selecting the appropriate wall formers and additives. One advantage of the template strategy, i.e. the production of the capsule starting with the formation of the very thin second layer as a template, is that in this production the components used as wall formers can be presented in the continuous water phase, which means that there is minimal contact with the core material during the formation of the shell. The components of the additional first layer can then be deposited as the first layer without interaction with the core material.

The microcapsule shells according to the invention have at least two layers, i.e. they can be, for example, two-layered, three-layered, four-layered, or five-layered. Preferably, the microcapsules have two or three layers.

According to one embodiment, the microcapsule has a third layer disposed on the outside of the first layer. In a further embodiment, the third layer is arranged on the outer side of the second layer. Preferably, in this embodiment, the second view is located on the outside of the first layer. This third layer can be used to customize the surface properties of the microcapsule for a particular application. This would include improving the adhesion of the microcapsules to a wide variety of surfaces and reducing agglomeration. The third layer also binds residual aldehydes, thus reducing the content of free aldehydes in the capsule dispersion. It can also provide additional (mechanical) stability or further increase impermeability. Depending on the application, the third layer may contain a component selected from amines, organic salts, inorganic salts, alcohols, ethers, polyphosphazenes and precious metals.

Precious metals increase the impermeability of the capsules and can impart additional catalytic properties to the microcapsule surface or the antibacterial effect of a silver layer. Organic salts, especially ammonium salts, lead to a cationization of the microcapsule surface, which causes it to adhere better to textiles, for example. Alcohols, when incorporated via free hydroxyl groups, also lead to the formation of H-bridges, which also allow better adhesion to substrates. An additional polyphosphazene layer or coating with inorganic salts, e.g. silicates, leads to an additional increase in impermeability without affecting biodegradability. According to a preferred embodiment, the third layer contains activated melamine. On the one hand, the melamine captures possible free aldehyde components of the first and/or second layer, increases the impermeability and stability of the capsule and can also influence the surface properties of the microcapsules and thus the adhesion and agglomeration behavior.

Due to the low wall thicknesses, the proportion of the second layer in the shell relative to the total weight of the shell is at most 30% w/w. For high biodegradability, the proportion is at most 25% w/w based on the total weight of the shell. More preferably, the proportion of the second layer is at most 20 wt. %. The proportion of the first layer in the shell relative to the total weight of the shell is at least 40% w/w, preferably at least 50% w/w, more preferably at least 60% w/w. The proportion of the third layer in the shell relative to the total weight of the shell is at most 25% w/w, preferably at most 20% w/w, more preferably at most 15% w/w.

The size of the microcapsules according to the invention is in the usual range for microcapsules. Thereby, the diameter can be in the range of 100 nm to 1 mm. The diameter depends on the exact capsule composition and the manufacturing process. The peak maximum of the particle size distribution is regularly used as a characteristic value for the size of the capsules. Preferably, the peak maximum of the particle size distribution is in the range of 1 μm to 500 μm. For example, the peak maximum of the particle size distribution may be 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, 120 μm, 140 μm, 160 μm, 180 μm 200 μm, 250 μm, 300 μm 350 μm, 400 μm, 450 μm or 500 μm. According to a particularly preferred embodiment, the microcapsules have a peak maximum particle size distribution of 10 μm to 100 μm. In particular, the peak maximum of the particle size distribution is in the range of 10 μm to 50 μm.

Product Containing Microcapsules

Due to the robustness or impermeability of this biodegradable capsule, it can be used in a variety of products. Consequently, according to a second aspect, the invention relates to a product containing microcapsules according to the first aspect wherein the product is selected from the group consisting of an adhesive system; a cosmetic product; a pharmaceutical product; a coating material, in particular a coated paper; a heat storage coating, for a self-healing coating or a corrosion coating; and coatings containing such microcapsules for functional packaging materials.

Further, in a third aspect, the invention relates to the use of microcapsules according to the first aspect for producing an article of manufacture according to the second aspect.

In other words, the microcapsules may be used in the manufacture of such a product. Consequently, the invention further relates to the use of the microcapsules according to the first aspect for the manufacture of the product, wherein the product is selected from the group consisting of an adhesive system; a cosmetic product; a pharmaceutical product; a coating material, in particular a coated paper; a heat storage coating, for a self-healing coating or a corrosion coating; and such microcapsule-containing coatings for functional packaging materials.

Manufacturing Method

Methods of making core/shell microcapsules are known to those skilled in the art. Generally, an oil-based core material that is insoluble or sparingly soluble in water is emulsified or dispersed in an aqueous phase containing the wall formers. Depending on the viscosity of liquid core materials, a wide variety of aggregates ranging from simple stirrers to high-performance dispersers are used to disperse the core material into fine oil droplets. In the process, the wall-formers from the continuous water phase are deposited on the oil droplet surface and can subsequently be crosslinked.

This mechanism is used in the in situ polymerization of amino and phenoplastic microcapsules and in the coacervation of water-soluble hydrocolloids.

In contrast, free radical polymerization uses oil-soluble acrylate monomers for wall formation. In addition, processes are used in which water-soluble and oil-soluble starting materials are reacted at the phase boundary of the emulsion droplets that form the solid shell.

Examples include the reaction of isocyanates and amines or alcohols to form polyurea or polyurethane walls (interfacial polymerization), and also the hydrolysis of silicate precursors with subsequent condensation to form an inorganic capsule wall (sol-gel process).

In a fourth aspect, the invention relates to a method for the production of microcapsules comprising a fragrance as core material and a shell consisting of three layers. Preferably, the very thin second layer serving as a diffusion barrier is provided as a template during the manufacturing process. Very small proportions of wall-formers of the type mentioned are required to build up this second layer. Preferably, after droplet formation at high stirring speeds, the sensitive templates are equipped with an electrically negative charge by suitable protective colloids (e.g. AMPS) in such a way that neither Ostwald ripening nor coalescence can occur. After this stable emulsion has been produced, the wall former, for example a suitable precondensate based on aminoplast resin, can be formed into a much thinner shell (layer) compared with the prior art at now greatly reduced stirring speeds. The thickness of the shell can be further reduced, in particular, by the addition of an aromatic alcohol, e.g. m-aminophenol. This is followed by the formation of a shell structure suitable for production, which unexpectedly shows good affinity for proteins such as gelatin or alginate when added and shows deposition on the templates without the expected problems such as gelation of the preparation, agglomeration formation and incompatibility of the structure donor.

The method comprises at least the following steps: (a) preparing an oil-in-water emulsion by emulsifying a core material in an aqueous phase, optionally with the addition of protective colloids; b) addition of the wall-forming component(s) of the inner shell layer, followed by deposition and curing, wherein the wall-forming component(s) of the inner shell layer are in particular an aldehydic component, an amine component and an aromatic alcohol; c) addition of the wall-forming component(s) of the middle shell layer, followed by deposition and curing, wherein the wall-forming components of the middle shell layer are in particular proteins and/or polysaccharides; and d) optionally adding the wall-forming component(s) of the outer shell layer, followed by deposition and curing, wherein the wall-forming component(s) of the outer shell layer is in particular an amine component.

Alternatively, steps a) and b) may be carried out as follows:
a) preparing an oil-in-water emulsion by emulsifying a core material in an aqueous phase in the presence of the wall-forming component(s) of the inner shell layer, optionally with the addition of protective colloids;
b) depositing and curing the wall-forming component(s) of the inner shell layer, wherein the wall-forming component(s) of the inner shell layer are in particular an aldehydic component, an amine component and an aromatic alcohol.

This process can be carried out either sequentially or as a so-called one-pot process. In the sequential process, only steps a) and b) are carried out in a first process until microcapsules with only the inner layer as shell (intermediate microcapsules) are obtained. In the following, a partial amount or the total amount of these intermediate microcapsules is then transferred to a further reactor. The further reaction steps are then carried out in this reactor. In the one-pot process, all process steps are carried out in a batch reactor. Carrying out the process without changing reactors is particularly time-saving.

For this purpose, the overall system should be adapted to the one-pot process. The correct choice of solids content, the correct temperature control, the coordinated addition of formulation components and the sequential addition of the wall-forming agents can be achieved in this way.

In one embodiment of the process, the process comprises preparing a water phase by dissolving a protective colloid, in particular acrylamidosulfonate and a methylated prepolymer in water. In this process, the pre-polymer is preferably produced by reacting an aldehyde with either melamine or urea. Optionally, methanol may be used in this process.

Furthermore, in the process according to the invention, mixing of the water phase may be carried out by stirring and adjusting a first temperature, wherein the first temperature is in the range of 30° C. to 40° C. Subsequently, an aromatic alcohol, in particular phloroglucin, resorcinol or aminophenol may be added to the water phase and dissolved therein.

Alternatively, in the process according to the invention, the preparation of an oil phase can be carried out by mixing a fragrance composition or a phase change material (PCM) with aromatic alcohols, in particular phloroglucin, resorcinol or aminophenol. Alternatively, reactive monomers or diisocyanate derivatives can be introduced into the fragrance composition. Subsequently, the adjustment of the first temperature can be carried out.

A further step can be the preparation of a two-phase mixture by adding the oil phase to the water phase and then increasing the speed.

Subsequently, emulsification can be started by adding formic acid. A regular determination of the particle size is recommended. Once the desired particle size has been reached, the two-phase mixture can be stirred further and a second temperature set for curing the capsule walls. The second temperature can be in the range of 55° C. to 65° C.

Subsequently, a melamine dispersion may be added to the microcapsule dispersion and a third temperature may be set, the third temperature preferably being in the range of 75° C. to 85° C.

Another suitable step is the addition of an aqueous urea solution to the microcapsule dispersion. To prepare the first shell, the microcapsule dispersion is added to a solution of gelatin and alginate.

In this case, cooling to 45° C. to 55° C. would follow, as well as adjusting the pH of the microcapsule dispersion to a value in the range of 3.7 to 4.3, in particular 3.9.

The microcapsule dispersion may then be cooled to a fourth temperature, the fourth temperature being in the range of 20° C. to 25° C. It may subsequently be cooled to a fifth temperature, the fifth temperature being in the range of 4° C. to 17° C., particularly 8° C.

Subsequently, the pH of the microcapsule dispersion would be adjusted to a value in the range of 4.3 to 5.1 and glutaraldehyde or glyoxal would be added. The reaction conditions, in particular temperature and pH, can be selected differently depending on the crosslinker. The skilled person can derive the appropriate conditions in each case, for example, from the reactivity of the crosslinker. The added amount of glutaraldehyde or glyoxal influences the crosslinking density of the first layer and thus, for example, the impermeability and degradability of the microcapsule shell. Accordingly, the skilled person can selectively vary the amount to adjust the property profile of the microcapsule. To generate the additional third layer, a melamine suspension consisting of melamine, formic acid and water can be prepared. This is followed by the addition of the melamine suspension to the microcapsule dispersion. Finally, the pH of the microcapsule dispersion would be adjusted to a value in the range of 9 to 12, in particular 10 toll.

In a fifth aspect, the invention relates to microcapsules having a fragrance as a core material and a shell comprising three layers prepared by a method according to the fourth aspect. In this regard, the middle layer comprises gelatin and alginate, the inner layer comprises melamine, formaldehyde and an aromatic alcohol, and the outer layer comprises melamine.

### EXAMPLES

### Example 1—Production of a Microcapsule According to the Invention with a Three-Layer Structure

1.1 Materials

TABLE 1
List of materials used for production

| | Concentration/ | Quantity/ |
|---|---|---|
| Stoffe | % | g |
| Lupasol PA140^{1),}* | 20 | 3,4 |
| Luracoll SD ^{2),}* | 67 | 1,6 |
| Water Addition 1 | 100 | 34,9 |
| Perfume oil Weiroclean * | 100 | 38,8 |
| Formic acid * Addition 1 | 20 | 0,5 |
| Resorcinol solution | 12,2 | 2,5 |
| Melafin Suspension^{3),6)} | 27 | 1,9 |
| Addition 1 | | |
| Urea solution | 16,6 | 4,7 |
| Water Addition 2 | 100 | 100,19 |
| Sodium sulfate* | 100 | 0,5 |
| Sodium alginate* | 100 | 1,4 |
| Pig skin gelatin* | 100 | 6,2 |
| Formic acid* Addition 2⁴⁾ | 20 | 1,4 |
| SodiumHydroxide | 20 | 0,8 |
| Addition 1 ⁴⁾ | | |
| Relugan GT50 ^{5),*} | 50 | 1,9 |
| Melafin Suspension^{3),6)} | 27 | 6,7 |
| Addition 2 | | |
| SodiumHydroxide | 20 | 2,2 |
| Addition 2 ⁴⁾ | | |

¹⁾Polymer based on: acrylamidosulfonate, source: BASF.
²⁾1,3,5-Triazine-2,4,6-triamine, polymer with formaldehyde, methylated (content (W/W): >= 60%-<= 80%), in water, Source: BASF
³⁾Cyanuric acid triamide (melamine); source: OCI Nitrogen BV
⁴⁾Addition depends on pH-value (see production process)
⁵⁾Glutaral; glutaraldehyde; glutardialdehyde (content (w/w): 50%), water (content (w/w): 50%), Source: BASF
⁶⁾Concentration based on acidified suspension.
*Quantities of the components refer to the commercial product and are used as supplied.

1.2 Manufacturing Process

To prepare Reaction Mixture 1, Lupasol PA140 and Luracoll SD with water Addition 1 were weighed into a beaker and premixed with a 4 cm dissolver disc. The beaker was fixed in a water bath, and stirred with the dissolver disc at 500 rpm at 30° C. until a clear solution was obtained.

Once the Luracoll/Lupasol solution was clear and reached 30-40° C., the perfume oil amount was slowly added while adjusting the speed (1100 rpm) to achieve the desired particle size. Then the pH of this mixture was acidified by adding Formic Acid Addition 1.

Emulsification was carried out for 20-30 min or extended accordingly until the desired particle size of 20-30 μm (peak-max) was achieved. The particle size was determined using a Beckmann-Coulter device (laser diffraction, Fraunhofer method). Once the particle size was reached, the speed was reduced to ensure gentle mixing.

Subsequently, the resorcinol solution was stirred in and performed under gentle stirring for 30-40 min. After the preforming time, the emulsion temperature was raised to 50° C. within 15 min. When this temperature was reached, the mixture was increased to 60° C. over a period of 15 min and this temperature was maintained for another 30 min. Melafin Suspension Addition 1 was then adjusted to a pH of 4.5 using 20% formic acid and added to the reaction mixture over a period of 90 min. The temperature was then maintained for 30 min. After the 30 min had elapsed, the temperature was first raised to 70° C. within 15 min. Then, within 15 min, the temperature was increased to 80° C. and maintained for 120 min. After that, the aqueous urea solution was added, the heat source was turned off, and the Reaction Mixture 1 was cooled to room temperature. In a separate beaker, sodium sulfate was dissolved in water while stirring with a wing stirrer at 40-50° C. Sodium alginate and porcine skin gelatin were slowly sprinkled into the heated water. After all solids were dissolved, Reaction Mixture 1 was added to the prepared gelatin/sodium alginate solution while stirring. When a homogeneous mixture was obtained, Formic Acid Addition 2 was used to adjust the pH to 3.9 by slow dripping, after which the heat source was removed. The mixture was then cooled to room temperature. After reaching room temperature, the reaction mixture was cooled with ice. When the temperature reached 8° C., the ice bath was removed and the pH was increased to 4.7 with Sodium Hydroxide Solution Addition 1. Relugan GT50 was then added. Care was taken to ensure that the temperature did not exceed 16-20° C. until Relugan GT50 was added.

Subsequently, the Melafin Suspension Addition 2, acidified to a pH of 4.5 using 20% formic acid, was slowly added. The reaction mixture was then heated to 60° C. and held for 60 min when the temperature was reached. After this holding time, the heat source was removed and the microcapsule suspension was gently stirred for 14 h. After the 14 h had elapsed, the microcapsule suspension was adjusted to a pH of 10.5 using Sodium Hydroxide Solution Addition 2.

1.3 Result

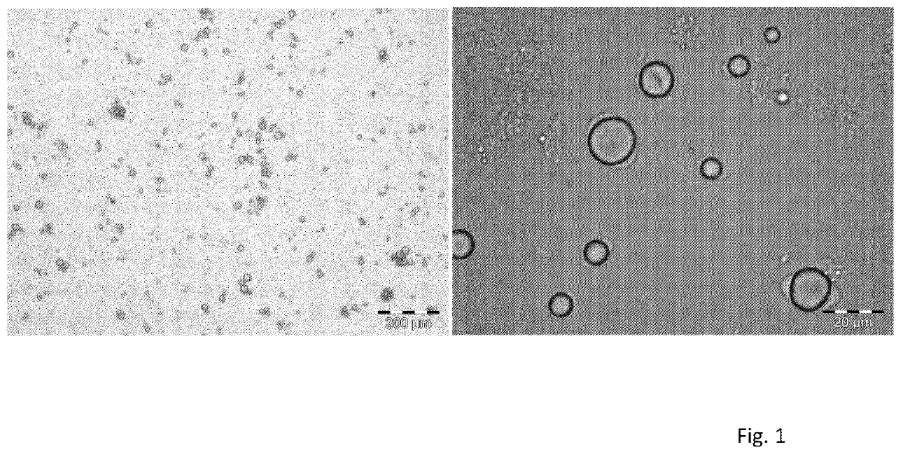

The obtained microcapsule MK 1 according to the invention was examined by light microscopy. Typical images are shown in FIG. 1. To evaluate MK 1, the pH, solids content, viscosity, particle size and nuclear material content in the slurry were determined. The result is shown in table 2.

TABLE 2
Analysis results of the microcapsule MK 1
according to the invention.

| | Measuring | |
|---|---|---|
| | method | MK 1 |
| pH | pH electrode | 8-10 |
| Solids content [%] | Mikro wave | 20-30 |
| | oven | |
| Viskosity | Brookfield- | <1000 |
| [mPas] | Viscometer | |
| Particle size peak | Laser | 20-30 |
| max. [μm] | diffraction | |
| Corematerial | Calculation | 15-20 |
| content in Slurry | from the recipe | |
| [%] | | |

### Example 2—Preparation of Reference Microcapsule not According to the Invention—Melamine-Formaldehyde Formulation

2.1 Materials

The materials used to prepare the Reference Microcapsules—Melamine Formaldehyde are shown in Table 3.

TABLE 3
List of substances used for production

| | | MK2 |
|---|---|---|
| Stoffe | Concentration*/% | Quantity/g |
| Lupasol PA140 ¹⁾ | 20 | 35,0 |
| Luracoll SD ²⁾ | 67 | 42,5 |
| Perfume oil weiroclean | 100 | 192,5 |
| Melafin^{3)’4)} Suspension | 27 | 48,8 |
| Urea solution | 28,6 | 70,0 |
| Deionized water for | 100 | 187,5 |
| emulsion | | |
| Formic acid | 10 | 8,8 |

¹⁾Polymer based on acrylamidosulfonate.
²⁾1,3,5-triazine-2,4,6-triamine, polymer with formaldehyde, methylated (content (w/w): >= 60%-<= 80%), in water
³⁾Melamine: Cyanuramide: 1,3,5-Trazine-2,4,6-triamine
⁴⁾Concentration based on acidified suspension.
*Quantities of the components refer to the commercial product and are used as supplied

2.2 Manufacturing Process (Based on Patent BASF EP 1 246693 B1)

Luracoll SD was stirred into deionized water and then Lupasol PA140 was added and stirred until a clear solution was obtained. The solution was heated to 30-35° C. in a water bath. While stirring with a dissolver disc, the perfume oil was added at 1100 rpm.

The pH of the oil-in-water emulsion was adjusted to 3.3-3.8 with a 10% formic acid. The emulsion was then stirred further at 1100 rpm for 30 min until a droplet size of 20-30 μm was achieved or extended accordingly until the desired particle size of 20-30 μm (peak-max) was reached. The particle size was determined using a Beckmann-Coulter device (laser diffraction, Fraunhofer method). The rotational speed was reduced as a function of viscosity to ensure good mixing. Stirring was continued at this speed for a further 30 min at 30-40° C. The emulsion was then heated to 60° C. and stirred further.

The melamine suspension was adjusted to a pH of 4.5 with formic acid (10%) and added to the reaction mixture. The mixture was kept at 60° C. for 60 min and then heated to 80° C. After stirring at 80° C. for 60 min, the urea solution was added.

After cooling to room temperature, the microcapsule dispersion was filtered through a 200 μm filter screen.

2.3 Result

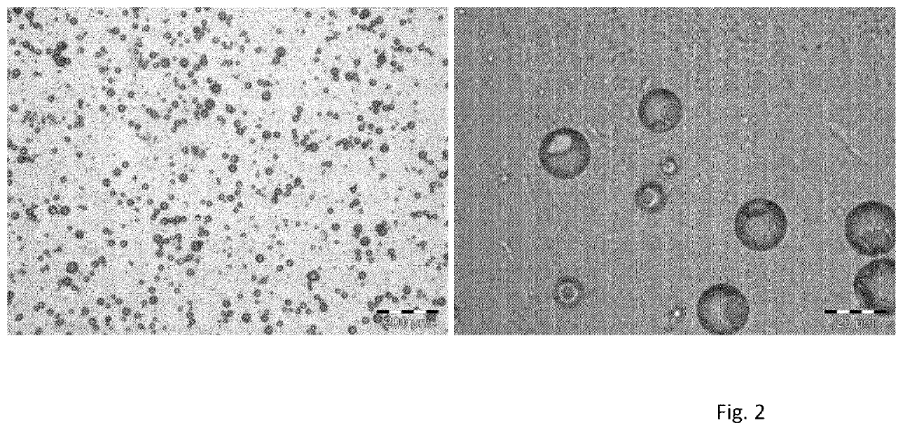

The obtained MF reference microcapsule MK 2 was examined by light microscopy. A typical image of MK 2 is shown in FIG. 2. To evaluate the obtained microcapsules, the pH, solids content, viscosity, particle size and nuclear material content in the slurry were determined. The result is shown in table 4.

TABLE 4
Analysis results of the reference microcapsule
MK 2 not according to the invention.

| | Measuring | |
|---|---|---|
| | method | MK2 |
| pH | pH electrode | 5,5-6,5 |
| Solids content [%] | Mikro wave | 37-41 |
| | oven | |
| Viskosity | Brookfield- | <1000 |
| [mPas] | Viscometer | |
| Particle size peak | Laser | 20-30 |
| max. [μm] | diffraction | |
| Core material | Calculation | 30-35 |
| content in Slurry | from the recipe | |
| [%] | | |

### Example 3—Production of Reference Microcapsules not According to the Invention—Gelatin/Alginate Formulation (Based on Patent Koehler DE 3424115)

3.1 Materials

The materials used for the production of the reference microcapsules—gelatin-alginate are shown in Table 5.

TABLE 5
List of materials used for the production
and amount used for the reference
microcapsule MK 3 not according to the invention.

| | Concentration*/ | MK3 |
|---|---|---|
| Stoffe | % | Quantity/g |
| Water Addition 1 | 100 | 204,4 |
| Sodium sulfate | 100 | 0,9 |
| Addition 1 | | |
| Sodium alginate | 100 | 2,9 |
| Pig skin gelatin | 100 | 12,7 |
| Sodium Hydroxide | 20 | 1,0 |
| Addition 1 ¹⁾ | | |
| Perfume oil | 100 | 79,6 |
| Weiroclean | | |
| Sodium sulfate | 100 | 0,8 |
| Addition 2 | | |
| Water Addition 2 | 100 | 180,5 |
| Acetic Acid | 96 | 2,7 |
| Sodium Hydroxide | 20 | 3.6 |
| Addition 2¹⁾ | | |
| Regulan GT50 ²⁾ | 50 | 3,9 |
| Sodium Hydroxide | 20 | 7,1 |
| Addition 3 ¹⁾ | | |

¹⁾ Addition depends on pH value (see manufacturing process).
²⁾ Glutaral; glutaraldehyde; glutardialdehyde (content (w/w): 50%), water (content (w/w): 50%).

3.2 Preparation Method

Sodium sulfate was weighed into an 800 ml beaker and dissolved by stirring with a paddle stirrer using water addition 1.

Perfume oil was weighed into a separate beaker and heated to 45° C. with stirring.

Sodium alginate and porcine skin gelatin were slowly sprinkled into the sodium sulfate solution and dissolved while stirring. Using Sodium Hydroxide Solution Addition 1, the pH was adjusted to 9.5.

To prepare an emulsion, the heated perfume oil was slowly added to the gelatin-alginate solution while increasing the stirrer speed to 1200 rpm. During emulsification, the droplet size was determined using a Beckmann-Coulter device (laser diffraction, Fraunhofer method). After a droplet size of 20-30 μm was reached, the speed was reduced so that gentle mixing was ensured.

In another beaker, Sodium Sulfate Addition 2 was dissolved using water Addition 2. Concentrated acetic acid was then added to this solution and heated to 45° C. with stirring.

The previously heated acetic acid/sodium sulfate solution was poured into a dropping funnel and added to the emulsion over a period of 15 min. The stirring speed was selected to ensure complete mixing.

After addition of the acetic acid solution, the mixture was stirred and cooled first to room temperature and then to 8° C. with ice.

When a suspension temperature of 8° C. was reached, the ice bath was removed and the pH was adjusted to 4.7 with Sodium Hydroxide Solution Addition 2. Regulan GT50 was then added. Care was taken to ensure that the temperature of the prepared suspension did not exceed 16-20° C. until Regulan GT50 was added.

Subsequently, the pH of the microcapsule suspension was adjusted to 10.5 while stirring by slowly adding the Sodium Hydroxide Solution Addition 3 (approx. 20-30 min).

3.3 Result

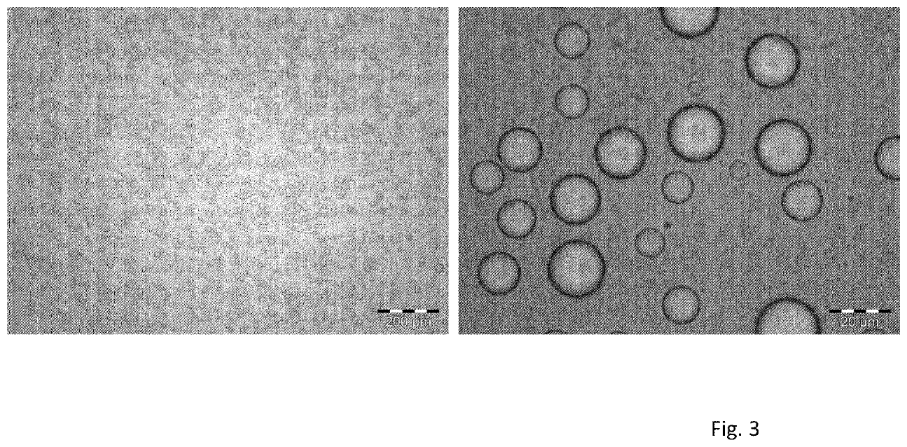

The obtained gelatin reference MK 3 microcapsules were examined by light microscopy. A typical image of MK 3 is shown in FIG. 3. To evaluate the obtained microcapsules, the pH, solid content, viscosity, particle size and nuclear material content in the microcapsule suspension were determined. The result is shown in Table 6.

TABLE 6
Analytical results of gelatin alginate reference
microcapsule MK 3.

| | Measuring method | MK3 |
|---|---|---|
| PH | pH electrode | 8-10 |
| Solids content [%] | Mikro wave oven | 18-22 |
| Viskosity | Brookfield-Viscometer | <1000 |
| [mPas] | | |
| Particle size peak | Laser diffraction | 20-30 |
| max. [μm] | | |
| Core material content | Calculation from the | 14-18 |
| in Slurry [%] | recipe | |

### Example 4—Production of the Further Microcapsule According to the Invention with a Three-Layer Structure

4.1 Materials

TABELLE 7
Liste derzur Herstellung verwendeten Stoffe

| | Konzentration/ | |
|---|---|---|
| Stoffe | % | Menge/g |
| Lupasol PA140 ^{1),}* | 20 | 3,4 |
| Luracoll SD ^{2),}* | 67 | 1.6 |
| Water Addition 1 | 100 | 34,9 |
| Perfume oil Weiroclean * | 100 | 38,8 |
| Formice Acid* Addition 1 | 20 | 0,5 |
| Resorcinol solution | 12,2 | 2,5 |
| Melafin Suspension^{3),6)} | 27 | 1,9 |
| Addition 1 | | |
| Urea solution | 16,6 | 4,7 |
| Water Addition 2 | 100 | 100,2 |
| Sodium sulfate* | 100 | 0,5 |
| Sodium alginate* | 100 | 1.4 |
| Pig skin gelatin* | 100 | 6.2 |
| Formic acid* Addition 2⁴⁾ | 20 | 1,4 |
| SodiumHydroxide | 20 | 0,8 |
| Addition 1 ⁴⁾ | | |
| Glyoxal 40% 5) | 40 | 1,4 |
| Melafin Suspension^{3),6)} | 27 | 6,7 |
| Addition 2 | | |
| SodiumHydroxide | 20 | 2,2 |
| Addition 2 ⁴⁾ | | |

¹⁾Polymer based on: acrylamidosulfonate, source: BASF.
²⁾1,3,5-triazine-2,4,6-triamine, polymer with formaldehyde, methylated (content (w/w): >= 60%-<= 80%), in water; source: BASF
³⁾Cyanuric acid triamide (melamine); source: OCI Nitrogen BV
⁴⁾Addition depends on pH-value (see manufacturing process)
⁵⁾Glyoxal; oxalaldehyde (content (w/w): 40 %), water (content (w/w): 60%), source: Sigma Aldrich
⁶⁾Concentration based on acidified suspension.
*Quantities of the components refer to the commercial product and are used as supplied

4.2 Preparation Procedure

For the preparation of reaction mixture 1, Lupasol PA140 and Luracoll SD with Water Addition 1 were weighed into a beaker and premixed with a 4 cm dissolver disc. The beaker was fixed in a water bath, and stirred with the dissolver disc at 500 rpm at 30° C. until a clear solution was obtained.

Once the Luracoll/Lupasol solution was clear and reached 30-40° C., the perfume oil amount was slowly added while adjusting the speed (1100 rpm) to achieve the desired particle size. Then the pH of this mixture was acidified by adding Formic Acid Addition 1.

Emulsification was carried out for 20-30 min or extended accordingly until the desired particle size of 20-30 μm (peak-max) was achieved. The particle size was determined using a Beckmann-Coulter device (laser diffraction, Fraunhofer method). Once the particle size was reached, the speed was reduced to ensure gentle mixing.

The resorcinol solution was then stirred in and preformed under gentle agitation for 30-40 min. At the end of the preforming time, the emulsion temperature was raised to 50° C. within 15 min. Upon reaching this temperature, the mixture was increased to 60° C. over a period of 15 min and this temperature was maintained for another 30 min. Subsequently, Melafin Suspension Addition 1 was adjusted to a pH of 4.5 using 20% formic acid and added to the reaction mixture over a period of 90 min. The temperature was then maintained for 30 min. After the 30 min had elapsed, the temperature was first raised to 70° C. within 15 min. Then, within 15 min, the temperature was increased to 80° C. and maintained for 120 min. After that, the aqueous urea solution was added, the heat source was turned off, and reaction mixture 1 was cooled to room temperature. In a separate beaker, sodium sulfate was dissolved in water while stirring with a wing stirrer at 40-50° C. Sodium alginate and porcine skin gelatin were slowly sprinkled into the heated water. After all solids were dissolved, Reaction Mixture 1 was added to the prepared gelatin/sodium alginate solution with stirring. When a homogeneous mixture was obtained, formic acid addition 2 was used to adjust the pH to 3.9 by slow dripping, after which the heat source was removed. The mixture was then cooled to room temperature. After reaching room temperature, the reaction mixture was cooled with ice. When the temperature reached 8° C., the ice bath was removed and the pH was increased to 4.7 with Sodium Hydroxide Solution Addition 1. The glyoxal solution was then added. Care was taken to ensure that the temperature did not exceed 16-20° C. until the glyoxal solution was added.

Subsequently, the Melafin Suspension, acidified to a pH of 4.5 by means of 20% formic acid, was added slowly (Addition 2). The reaction mixture was then heated to 60° C. and held for 60 min when the temperature was reached. After this holding time, the heat source was removed and the microcapsule suspension was gently stirred for 14 h. After the 14 h had elapsed, the microcapsule suspension was adjusted to a pH of 10.5 using Sodium Hydroxide Addition 2.

4.3 Result

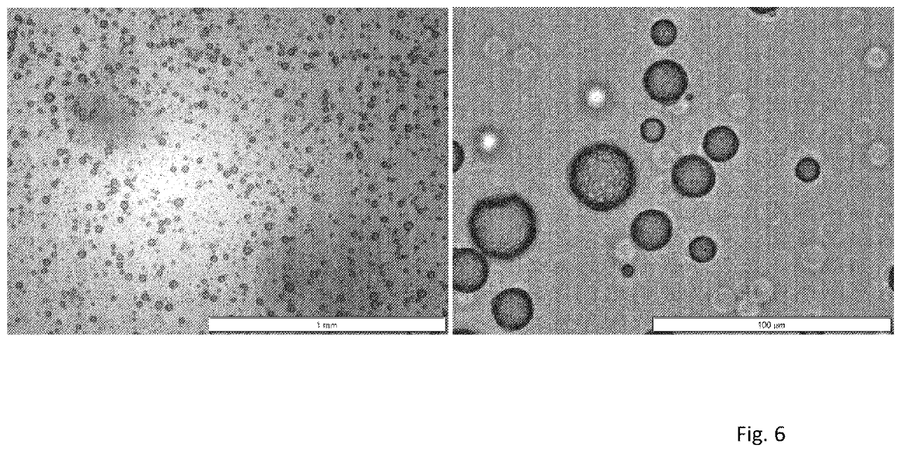

The obtained microcapsule MK 4 according to the invention was examined by light microscopy. Typical images are shown in FIG. 6. To evaluate MK 4, the pH, solids content, viscosity, particle size and nuclear material content in the slurry were determined. The result is shown in table 8.

TABLE 8
Analytical results of the microcapsule MK4
according to the invention

| | Measuring | |
|---|---|---|
| | method | MK 4 |
| PH | pH electrode | 8-10 |
| Solids content [%] | Mikro wave | 20-30 |
| | oven | |
| Viskosity | Brookfield- | < 1000 |
| [mPas] | Viscometer | |
| Particle size peak | Laser | 20-30 |
| max. [μm] | diffraction | |
| Corematerial | Calculation | 15-20 |
| content in Slurry | from the recipe | |
| [%] | | |

### Example 5—Stability Measurement of the Microcapsules

5.1 Preliminary Remark

To determine the stability of microcapsules, they were stored in a model fabric softener formulation at 40° C. for a period of up to 12 weeks and the concentration of fragrance diffused from the capsule interior into the surrounding formulation was determined by HS-GC/MS. Based on the measured values, the residual amount of perfume oil still present in the capsule was calculated.

Model fabric softener formulation based on Rewoquat WE 18 E US from Evonik following the formulation from the associated product data sheet:

To prepare the fabric softener base, 94 g water was heated to 50° C. and 5.65 g Rewoquat WE 18 E US was added to the heated water while stirring. The mixture was cooled to room temperature, then the microcapsule dispersion was added.

5.2 Experimental Procedure

For this purpose, the microcapsule suspension (slurry) was carefully homogenized and stored at a concentration of 1 wt % in the model formulation at 40° C., hermetically sealed, in the heating cabinet. The non-encapsulated fragrance with analogous fragrance concentration in the model formulation served as a comparison.

After predetermined storage time, the samples were removed from the warming cabinet and an aliquot was weighed into a 20 ml headspace vial. The vial was then immediately sealed.

These samples were evaluated by headspace solid-phase micro-extraction (SPME) using capillary gas chromatography and, after detection with a mass selective detector (MSD).

5.3 Results

The stability curve of the capsule of the invention according to Examples 1 and 4 and of the reference capsule according to Example 2 over 12 weeks is shown in Table 9.

TABELLE 9
Measured values of the stability test

| | Capsules | | |
|---|---|---|---|
| | according | | |
| | to the | Capsules | Melamine- |
| | invention | according to | Formaldehyde |
| Storage | (example | the invention | Capsules |
| duration | 1) | (example 4) | (Example 2) |
| Week | % Stability | % Stability | % Stability |
| 0 | 100 | 100 | 100 |
| 1 | 92 | 87 | 100 |
| 4 | 59 | 52 | 87 |
| 12 | 22 | 20 | 20 |

As can be seen from Table 9, the microcapsules MK 1 and MK 4 according to the invention show comparable stability to the MF reference microcapsule MK 2 after 12 weeks of storage in a model formulation.

The gelatin/alginate reference microcapsule MK 3 shows no capsule stability in the test medium under the selected test conditions (disintegration already during sample preparation), so that it was not possible to record measured values for stability assessment here within the required time frame.

For the calculation of the capsule stability, a change in concentration of 16 individual ingredients of the encapsulated fragrance was considered. A decrease in stability results in a leakage of the encapsulated fragrance, which can subsequently be detected by gas chromatography using headspace SPME. Since all capsule dispersions were adjusted to a defined oil content of 15 w/w %, a direct comparison of the capsule samples investigated is possible. Individual ingredients (or their individual signals detected by gas chromatography) which, due to fluctuations caused by measurement technology, indicate higher concentrations than were theoretically possible in comparison with the reference standard, were only taken into account in the evaluation up to the theoretical maximum concentration.

### Example 6—Biodegradability Measurement of Microcapsules (According to OECD 301 F)

6.1 General

The purpose of this test is to evaluate the rapid biodegradability of the microcapsules.

The standard test concentration of the samples to be tested is 1000 mg/I O₂. The measuring heads and the controller measure the oxygen consumption in a closed system. The consumption of oxygen and the simultaneous binding of resulting carbon dioxide to sodium hydroxide pellets creates a negative pressure in the system. The measuring heads register and store this pressure over the set measuring period. The stored values are read into the controller via infrared transmission. They can be transferred to a PC and evaluated using the Achat OC program.

To eliminate the influence of the core material for degradation, perfluorooctane was encapsulated (degradation rate=<1%).

6.2 Equipment and Chemicals
Instruments: OxiTop-Control measuring system, WTW incl. controller OxiTop OC 110 with interface cable for PC, 6 measuring heads OxiTop C, 6 glass vials with a total volume of 510 ml each, 6 magnetic stirrers, 6 rubber quivers, 1 magnetic stirring system, and readout software Achat OC
   Drying oven ORI-BSB, set to 20° C.
   Outflow stones Oxygenius, 30×15×15 mm³
   Aquarium aeration pump Thomas-ASF No. 1230053
   Filter chute D=90 mm
   Suction bottle 2 I
   White belt filter MN 640 d, D=90 mm, Fa. Macherey+Nagel Manual “Oxi Top Control System”, WTW company
Chemicals: Activated sludge from the factory's own or a municipal wastewater treatment plant
   Ethylene glycol z.A., Merck company
   Reference sample with COD 1000 mg/I O₂
   Walnut shell flour, Senger Naturrohstoffe company
   Nutrient salt solution from the factory's own or a municipal wastewater treatment plant
   Caustic soda cookie z.A.>99%, Fa. Merck
   Cell test CSB LCK 514, Dr. Lange company

6.3 Performance

6.3.1 Preparation of the Microcapsule Slurries

The microcapsules MK 1 to MK 4 were prepared according to the descriptions of Examples 1 to 4, with the difference that instead of the perfume oil, the completely persistent perfluorooctane (degradation rate <1%) was used as core material. This eliminates any influence of the core material on the experimental result.

6.3.2 Sample Preparation

For the 28-day degradation tests, the microcapsule slurries were used as obtained from the fabrication.

In the case of the extended degradation tests over 60 days, the microcapsule slurries were washed in water after preparation by centrifuging and redispersing three times to separate dissolved residues. For this, a sample of 20-30 mL is centrifuged at 12,000 rpm for 10 minutes each time. After aspiration of the clear supernatant, make up with 20-30 mL of water and redisperse the sediment by shaking.

6.3.3 Preparation of the Reference Sample

711.6 mg ethylene glycol was dissolved in a 1 L volumetric flask and filled up to the mark. This corresponds to a COD of 1000 mg/l O₂. Ethylene glycol is considered to have good biodegradability and is used here as a reference.

Due to the rapid degradation of ethylene glycol, walnut shell flour was added as another reference for the extended 60-day test. Walnut shell flour consists of a mixture of biopolymers, especially cellulose and lignin, and serves as a solid-based biobased reference. Due to the slow degradation of walnut shell flour, the test progress can be followed over the complete period of 60 days. For this purpose, 117.36 g of walnut shell flour was homogeneously dispersed in 11 of water under stirring. Aliquots of this mixture were taken under stirring for COD determination. Based on the mean COD value of 1290±33 mg/l O₂, the required feed amount was calculated and transferred to the OxiTop bottles with stirring.

6.3.4 Preparation of the Biosludge

Activated sludge was taken from the outlet of the activated sludge tank of a factory-owned or a municipal wastewater treatment plant using a 20 I bucket. After settling for 30 minutes, the supernatant water was discarded.

Subsequently, the concentrated biosludge in the bucket was permanently aerated for 3 days using the aquarium pump and an outflow stone.

6.3.5 Dry Content Determination of the Biosludge

After 3 days, 100 ml of the concentrated biosludge was filtered off using a filter chute through a white belt filter. The filter cake is dried for 24 h at 105° C. in a drying oven.

$$T G = \frac{A}{E}$$

TG=Dry content of the biological sludge in %.

E=Weighing of the filter cake in g

A=Weighing out of the filter cake in g

c=TG×10

c=concentration of the biosludge in g/I

6.3.6 Adjustment of the Samples to a COD of 1000 mg/l O₂

The COD value of the samples to be analyzed was determined with the cuvette test COD LCK 514. The sample is diluted with water until the COD value of 1000 mg/I O₂ is reached.

6.3.7 Preparation of the Mixtures

For one sample, 6 OxiTop bottles were used, since duplicate determinations are carried out in each case.

In each case 2 bottles (double determination) were used for the following measurements:
Biodegradability of the sample
Biodegradability of the ethylene glycol solution (=reference solution)
Blank sample (=determination of the residual degradation of the sludge itself).

For each bottle are required
25 ml sample with a COD of 1000 mg/l O₂ (for blank sample 25 ml distilled water).
3.5 ml nutrient solution
44.5 mg otro (oven-dry) sludge
distilled water to fill up to a total volume of 250 ml.

3 sodium bicarbonate pellets were placed in each rubber quiver with a spatula. After adding sample, nutrient solution, biosludge and distilled water to the bottles, a magnetic stir bar was placed in each bottle. Then, the rubber holes were placed on the respective bottle necks and the measuring heads were screwed tightly onto the bottles.

6.4 Measurement and Evaluation

The programming of the OxiTop-C measuring heads and the evaluation of the data are described in detail in the manual “OxiTop Control System”, WTW.

6.5 Result

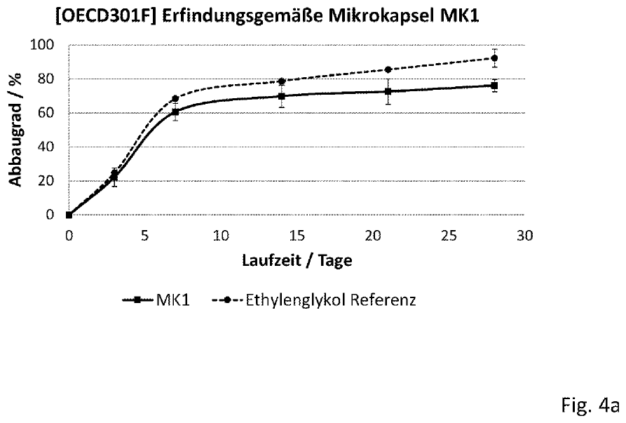

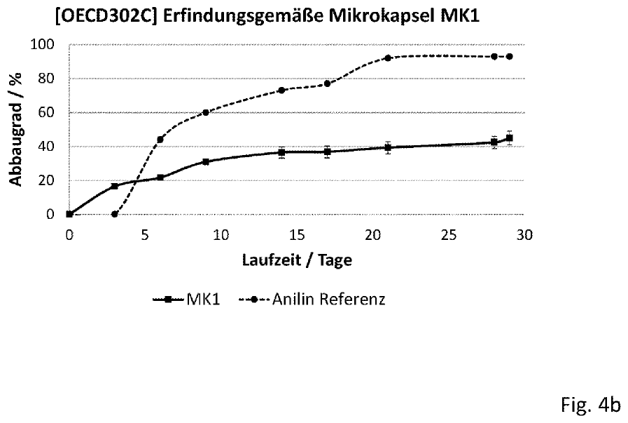

The biodegradation diagram after 28 days of the capsule MK 1 according to OECD 301 F is shown in FIG. 4(a).

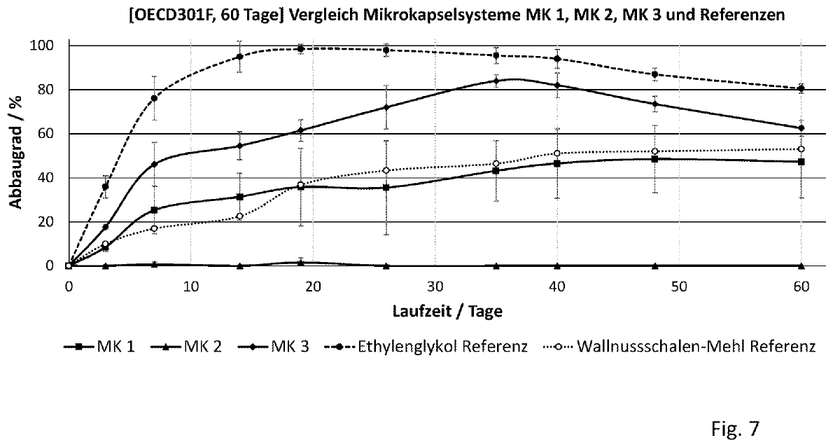

The capsule MK 1 according to the invention shows a biodegradability of 76±4% after 28 days. Furthermore, capsule MK 4 according to the invention shows a biodegradability of 78±9% after 28 days. After washing, the capsule MK 1 according to the invention shows a biodegradability of 47±16% after 60 days. FIG. 7 shows a comparison of the biodegradability measurement according to OECD 301F. Here it can be seen that the microcapsule MK1 according to the invention has a comparable biodegradability to the natural-based reference walnut shell flour with a biodegradability of 53% after 60 days.

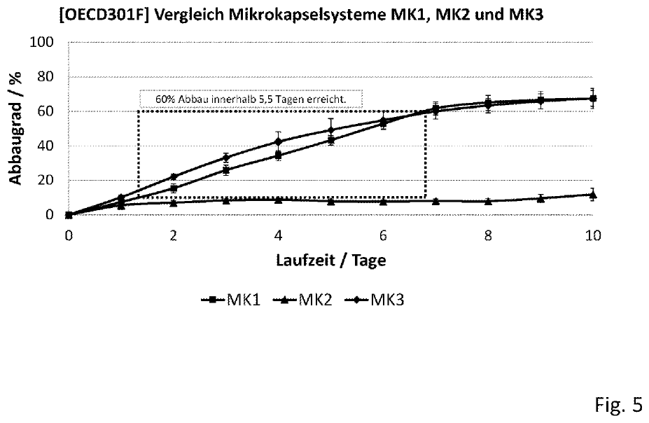

FIG. 5 shows a comparison of biodegradability measurements according to OECD301F between the microcapsule MK 1 according to the invention, the MF reference microcapsule MK 2, and the gelatin/alginate reference microcapsule MK 3. The OECD301F specification (according to “Revised Introduction to the OECD Guidlines for testing of Chemicals, section 3, Part 1, dated 23 Mar. 2006”) stipulates that the substance to be tested must reach a biodegradation level of 60% within a 10-day time window (starting from a degradation of 10%). Both the microcapsule MK 1 according to the invention and the gelatin/alginate reference microcapsule MK 3 show very rapid biodegradation compared to the MF reference microcapsule MK 2. The required time period for a degradation of 60% is already reached after 7 days.

It can be seen that, in line with experience, the degree of degradation of the standard MF capsules MK 2 reaches the range of 10% within a short time and forms a plateau here, indicating no further degradation within the measurement period.

The cross-linked gelatin-alginate microcapsules MK 3 prove to have good biodegradability according to experience. They reach the value of 68±5% within 10 days.

The microcapsule MK 1 according to the invention also shows a degradation degree of 68±6% after 10 days.

FIG. 7 shows the comparative degradation curves of the MK 1 according to the invention, the reference MK 2 and MK 3 as well as the reference substances ethylene glycol and walnut shell flour. It can be seen that the rapidly biodegradable reference sample ethylene glycol reaches the maximum degradability between the 15th day and the 25th day of the measurement. Thereafter, the measured value apparently drops, caused by processes of the inoculum due to the absence of a degradable food source. This effect can be evaluated as a measurement artifact. A comparable behavior is evident for the easily degradable reference capsule MK 3. The maximum of degradability is reached for sample MK 3 between the 25th day and the 45th day of measurement and drops thereafter in the same way. The poorly degradable reference capsule MK 2 shows no biodegradability during the course of the measurement. Negative measured values (which occurred in particular in the second half of the measurement period) were set to zero. The natural-based reference walnut shell flour shows the typical step-like degradation of a complex mixture of substances. The maximum biodegradability is reached in the range of the 40th day of the measurement, and this value remains constant within the range of variation until the end of the measurement after 60 days. A similar degradation behavior can be observed for the microcapsule MK 1 according to the invention. Over a step-like progression, a mean degradation level of 47% is achieved after 60 days, with an absolute range of variation between 30 and 65% biodegradability.

TABLE 10
Representation of the degradation values according to OECD 301 (60 days)

| | Method | 7 days | 14 days | 26 days | 40 days | 48 days | 60 days |
|---|---|---|---|---|---|---|---|
| Capsule (MK1) | OECD | 25 ± 11 | 31 ± 10 | 36 ± 21 | 47 ± 16 | 49 ± 15 | 47 ± 16 |
| according to the | 301 F | | | | | | |
| the invention, washed1) | | | | | | | |
| Melamine formaldeyh | OECD | 1 ± 1 | 0 ± 0 | 0 ± 0 | 0 ± 0 | 0 ± 0 | 0 ± 0 |
| d capsules | 301 F | | | | | | |
| (MK 2), | | | | | | | |
| washed2) | | | | | | | |
| Gelatin alginate | OECD | 46 ± 10 | 55 ± 6 | 72 ± 10 | 82 ± 6 | 74 ± 4 | 63 ± 4 |
| capsules (MK 3), | 301 F | | | | | | |
| washed3) | | | | | | | |
| Walnut shell | OECD | 17 | 23 | 43 | 51 | 52 | 53 |
| flour4) | 301 F | | | | | | |

1) MK1, quadruple determination
2) MK2, double determination
3) MK3 double determination
4) walnut shell flour reference, single determination

Finally, it should be expressly noted that the above-described examples of embodiments of the device according to the invention serve only to discuss the claimed teaching, but do not limit it to the examples of embodiments.

### Example 7—Biodegradability Measurement of Microcapsules (According to OECD 302 C)

7.1 General

The purpose of this test is to assess the basic biodegradability of the microcapsules.

The measurement was carried out on the basis of the specifications according to OECD302C 1981-05 by a testing laboratory accredited according to DIN EN ISO 17025 (SGS Institut Fresenius GmbH, Taunusstein, Germany). The modified test procedure with natural inoculum and modified detection method (direct quantification of the carbon dioxide formed) was used.

Analogous to the sample preparation for the biodegradability measurement according to OECD 301 F (see Example 5), microcapsule lurries were prepared containing perfluorooctane (degradation rate=<1%) as core material. Thus, an influence of the core material on the biodegradability of the microcapsules is prevented.

7.2 Equipment and Chemicals

According to the information provided by the test laboratory, the inoculum used consists of activated sludge from the Taunusstein-Bleidenstadt wastewater treatment plant (— 100 mg dry weight equivalent/L batch). Aniline was used as the control.

7.3 Procedure

First, a sample was taken from the respective microcapsule slurries and an analysis of the total organic carbon (TOC) was performed. By knowing the molar ratio of carbon dioxide to elemental carbon, the TOC made it possible to calculate the theoretical amount of carbon dioxide that can be released during the degradation of the test substance (TCO₂, “theoretical amount of CO₂”).

The test preparations were prepared in a volume of 3500 mL each. The test substance and the inoculum were incubated in this volume at room temperature in a mineral nutrient medium. By knowing the TOC of the microcapsule slurry, a carbon concentration of about 25 mg C/L was set. Thus, only the carbon from the test article was available as an energy source for the microorganisms present in the inoculum. The test mixtures were aerated with CO₂-free compressed air and stirred by magnetic stirrers. As the test article was degraded by microorganisms, the carbon contained was converted into carbon dioxide. This gas evolution was collected by means of gas wash bottles mounted on the test specimen. The gas wash bottles were filled with a solution of barium hydroxide, which binds the carbon dioxide produced. Titration with hydrochloric acid allows the carbon dioxide formed in the test batch to be quantified. The degree of degradation of the test substance was then calculated by comparing the theoretically producible carbon dioxide (from the TOC measurement) with the amount of carbon dioxide actually determined. Three preparations were made for each test substance, which made it possible to determine an average degree of degradation.

In order to determine the amount of carbon dioxide produced by the inoculum, two so-called blank samples were determined in parallel to the test batch, which did not contain any test substance but only the inoculum. The amount of carbon dioxide determined in this way was subtracted from the test sample.

In analogy to the described procedure, an additional preparation with a control substance (aniline) and a preparation with a mixture of the test substance and the control substance (toxicity control) were prepared and carried along.

The duration of the test was 28 days and 60 days, respectively. On the last day, the degradation test was stopped by the addition of conc. hydrochloric acid and the carbonates or dissolved carbon dioxide in the preparation were expelled and also quantified in the connected gas wash bottles.

7.4 Measurement and Evaluation

After titrating the barium hydroxide solution in the gas wash bottles, the amount of carbon dioxide produced in the test batch can be quantified and the degree of degradation of the test substance can be calculated using the following formula:

$$\% degradation = \frac{mg {CO}_{2} p roduced ⋆ 100}{( mg test substance in the batch ) ⋆ {TCO}_{2}}$$

7.5 Result

TABLE 11
Representation of the degradation values according to OECD 301 F and OCED 302 C (28 days)

| | Method | 3 days | 7 days | 10 days | 14 days | 21 days | 28 days |
|---|---|---|---|---|---|---|---|
| Capsule | OECD | 22 ± 5 | 61 ± 5 | 68 ± 6 | 70 ± 7 | 73 ± 8 | 76 ± 4 |
| according to | 301 F | | | | | | |
| invention | OECD | 16 ± 1 | 22 ± 1 | 31 ± 1 | 37 ± 3 | 39 ± 4 | 45 ± 4 |
| (MK 1) | 302 C | | | | | | |
| Capsule | OECD | 23 ± 6 | 64 ± 8 | 69 ± 5 | 72 ± 7 | 74 ± 9 | 78 ± 8 |
| according to | 301 F | | | | | | |
| the | | | | | | | |
| invention | | | | | | | |
| (MK4) | | | | | | | |
| Melamine | OECD | 8 ± 1 | 8 ± 1 | 12 ± 4 | − | − | − |
| formaldeyh | 301 F | | | | | | |
| d capsules | | | | | | | |
| (MK 2) | | | | | | | |
| Gelatin alginate | OECD | 33 ± 3 | 60 ± 5 | 68 ± 5 | − | − | − |
| capsules (MK 3) | 301 F | | | | | | |

The biodegradation diagram according to OECD302C of the capsule MK1 according to the invention is shown in FIG. 4(b).

The capsules MK 1 according to the invention show a degradability value of 45±4% after 28 days.

Finally, it should be expressly noted that the above-described embodiments of the device according to the invention serve only to explain the claimed teaching, but do not restrict it to the embodiments.

1. Microcapsules for use in a high-demand area, selected from detergents and cleaning agents, cosmetic products, adhesive systems, paints and dispersions, coating materials, comprising a core material and a shell, wherein the shell consists of at least a first and a second layer whose chemical compositions differ, and wherein the shell has a biodegradability measured according to OECD 301 F of at least 40%.

2. The microcapsules according to claim 1, wherein the shell has a biodegradability measured according to OECD 301 F of at least 50%, preferably at least 60%, more preferably at least 70%.

3. The microcapsules according to claim 1, wherein the microcapsule has a impermeability ensuring a leakage of at most 80% w/w of the core material used after storage for a period of 12 weeks at a temperature of 0 to 40° C., preferably at most 75% w/w, more preferably at most 70% w/w.

4. The microcapsules according to claim 1, wherein the first layer contains one or more biodegradable components, wherein the biodegradable components are selected from the group consisting of proteins such as gelatin; polysaccharides such as alginate, gum arabic, chitin, or starch; phenolic macromolecules such as lignin; polyglucosamines such as chitosan, polyvinyl esters such as polyvinyl alcohols and polyvinyl acetate; phosphazenes and polyesters such as polylactide or polyhydroxyalkanoate, wherein the first layer contains in particular gelatin and/or alginate.

5. The microcapsules according to claim 4, wherein the first layer comprises one or more curing agents, preferably selected from the group consisting of an aldehyde such as glutaraldehyde, formaldehyde or glyoxal, a tannin, an enzyme such as transglutaminase and an organic anhydride such as maleic anhydride, wherein the curing agent is particularly preferably glutaraldehyde or glyoxal.

6. The microcapsules according to claim 1, wherein the first layer comprises one or more inorganic components, in particular inorganic salts such as calcium carbonate or polysilicates.

7. The microcapsules according to claim 1, wherein the second layer is composed of one or more components selected from the group consisting of an aldehydic component, an aromatic alcohol, an amine component, an acrylate component and an isocyanate component.

8. The microcapsule according to claim 7, wherein the second layer contains an aldehydic component selected from the group consisting of formaldehyde, glutaraldehyde, succinaldehyde, furfural, and glyoxal, and preferably the percentage of the aldehydic component for polycondensation based on the total weight of the second shell is in the range of 5 to 50% w/w, preferably in the range of 10 to 30% w/w, more preferably in the range of 15 to 20% w/w.

9. The microcapsules according to claim 7, wherein the second layer comprises an aromatic alcohol selected from the group consisting of resorcinol, phloroglucin and aminophenol, and preferably the percentage of the aromatic alcohol based on the total weight of the second layer is in the range of 1.0 to 20% w/w, preferably in the range of 5 to 15% w/w, more preferably in the range of 9 to 13% w/w.

10. The microcapsules according to claim 7, wherein the second layer comprises an amine component selected from the group consisting of melamine, melamine derivatives and urea and combinations thereof, and preferably the percentage of the amine component based on the total weight of the second layer is in the range of from 20% w/w to 85% w/w, preferably in the range of from 40% w/w to 80% w/w, more preferably in the range of from 55% w/w to 70% w/w.

11. The microcapsules according to claim 1, wherein the second layer is arranged on the inner side of the first layer.

12. The microcapsules according to claim 1, wherein the percentage of the second layer on the shell relative to the total weight of the shell is at most 30% w/w, preferably at most 25% w/w, more preferably at most 20% w/w.

13. The microcapsules according to claim 1, wherein the second layer has an average thickness in the range of 0.01 μm to 1 μm, preferably 0.02 μm to 0.5 μm, particularly preferably 0.05 μm to 0.30 μm.

14. The microcapsules according to claim 1, wherein the microcapsule has a third layer which is arranged on the outside of the first layer and which contains a component selected from amines, organic salts, inorganic salts, alcohols, ethers, polyphosphazenes, and noble metals, wherein the proportion of the third layer in the shell relative to the total weight of the shell is at most 35%, preferably at most 25% by weight, particularly preferably at most 15% by weight.

15. The microcapsules according to claim 1, wherein the core material is selected from the group consisting of fragrances, flavors, phase change materials, cosmetic active ingredients, pharmaceutical active ingredients, catalysts, initiator systems, adhesive components and hydrophobic reactive components.

16. An article comprising microcapsules according to claim 1, wherein the article is selected from the group consisting of an adhesive system; a cosmetic product; a pharmaceutical product; a coating material, in particular a coated paper; a heat storage coating, a self-healing coating or a corrosion coating; and such microcapsule-containing coatings for functional packaging materials.

17. A process for preparing microcapsules according to claim 1, characterized by the following steps:
(a) preparing an oil-in-water emulsion by emulsifying a core material in an aqueous phase in the presence of the wall-forming component(s) of the inner, second shell layer, optionally with the addition of protective colloids;
b) depositing and curing the wall-forming component(s) of the inner, second shell layer, wherein the wall-forming component(s) of the inner, second shell layer are in particular an aldehydic component, an amine component and an aromatic alcohol;
c) addition of the wall-forming component(s) of the middle, first shell layer, followed by deposition and curing, wherein the wall-forming component(s) of the middle, first shell layer are in particular proteins and/or polysaccharides; and
d) optionally adding the wall-forming component(s) of the outer, third shell layer, followed by deposition and curing, wherein the wall-forming component(s) of the outer, third shell layer is in particular an amine component.

18. The process according to claim 17, wherein the process steps are carried out in a reactor.
